(19) United States
(12) Patent Application Publication
(10) Pub. No.: US 2024/0238584 A1
(43) Pub. Date: Jul. 18, 2024

(54) Improvements Relating To Functional Electrical Stimulation Garments

(71) Applicant: MYGO4WARD LTD, Forres (GB)

(72) Inventors: David Powney, Forres (GB); Judith Simms, Forres (GB)

(21) Appl. No.: 18/272,042

(22) PCT Filed: Jan. 10, 2022
(86) PCT No.: PCT/GB2022/050042

§ 371 Date: Jul. 12, 2023

(30) Foreign Application Priority Data
Jan. 13, 2021 (GB) 2100423.9

(51) Int. Cl. A61N 1/04; A61N 1/36

(52) U.S. Cl. CPC A61N 1/0484; A61N 1/0452; A61N 1/36003; A61N 1/36034

(57) ABSTRACT

The invention relates to apparatus and method(s) for facilitating walking using functional electrical stimulation. In various aspects, apparatus(es) and method(s) for eliciting or enhancing motion of body part(s) in a human or animal body are described. An apparatus for facilitating walking in a human body comprising: a garment configured to be worn in a close fit on a leg of a human body, the garment comprising: at least one flexible, articulating portion configured to span a joint of a leg (e.g. one or more of the hip, knee, ankle and joints; a flexible, resilient fabric substrate having an inner surface for engaging a leg closely when worn; and, on the inner surface of the substrate, an electrode arrangement comprising: at least two pairs of conductive, flexible, printed electrodes configured with respect to the articulating portion for electrically stimulating a respective (or the same) predetermined muscle group with respect to the joint; further in which the electrode arrangement comprises a first pair of electrodes configured to stimulate a first (e.g. the tibialis anterior) muscle group; and, at least one second pair of electrodes configured to stimulate a second (e.g. the quadricep) muscle group or the first muscle group.

### FIELD OF THE INVENTION

The invention relates to apparatus(es) and method(s) for facilitating walking by eliciting or enhancing motion using functional electrical stimulation. In a further aspect, apparatus and method(s) for eliciting or enhancing motion of body part(s) in a human or animal body are described.

### BACKGROUND

CN109157743 SHAO describes a training garment and a training method for simulating human weight bearing through biological electric current, in this case also having a pre-heating function. This is treatment to build muscle.

WO2011080263 MAUGERI describes a training/medical low-frequency electromyostimulating garment aimed at treatment, in this case rehabilitation.

U.S. Pat. No. 6,453,202 YAMAZAKI describes electrode tights for a shape-up apparatus for treatment.

US2002077688 and US2002077689A1 KIRKLAND describe an electrode-positioning body garment also aimed at treatment.

WO2012/116407 SOUTHWELL describes a transcutaneous stimulation method and system for treating a lower pelvic region.

WO2010/084391 CROWE describes a method and apparatus for stimulating pelvic floor muscles.

CN111195394 YUAN describes an electromyographic electrical stimulation device aimed at facilitating walking using a rigid external cuff for locating electrodes on a limb.

US2019217091 MOHAMMED describes a stimulation device comprising spaced apart electrodes for activating at least one muscle involved in raising the foot to aid walking and appears to aim at avoiding a foot sensor.

CN108392737 LI describes a multi-channel functional electrical stimulation output control method for lower limb rehabilitation of myoelectric modulation and uses spaced apart electrodes on the leg to aid walking.

US2018140842 OLAIGHIN describes apparatus for management of a Parkinson's disease patient's gait; the electrodes are skin mounted (see paragraph 174) or implanted (see paragraph 208).

CN105536146 YE describes a multi-channel electrical stimulation lower limb walking aid and method.

EP3097947 MIHARA uses electrodes to stimulate walking and describes an electrostimulator controller for determining initial stance, midstance, terminal stance, initial swing, or terminal swing on the basis of detection signals; and provides electrodes for outputting electrical stimulation to muscles.

WO9209328 GRAUPE describes a stimulation system (100) for providing upper-motor-neuron paralyzed patients with capabilities of unbraced standing and unbraced walking.

US2014128939 and WO2013063200A1 EMBREY describe a functional electrical stimulation (FES) method and system to improve walking and other locomotion functions.

US2017/0182320 KOLB describes a wearable pulse generator device.

WO2008/088985 HARRY describes a system method for neuro-stimulation using a wearable device.

How It Works—Wearable Tech Company_Wearable X (downloaded from https://www.wearablex.com/pages/how-it-works_v0) describes NADI X Yoga pants that have haptic detection sensors that vibrate to provide feedback in posing.

WO2019110595A1 LAY (Atlantic Therapeutics) describes a conductive circuit and uses screen printed electrodes in shorts to treat incontinence.

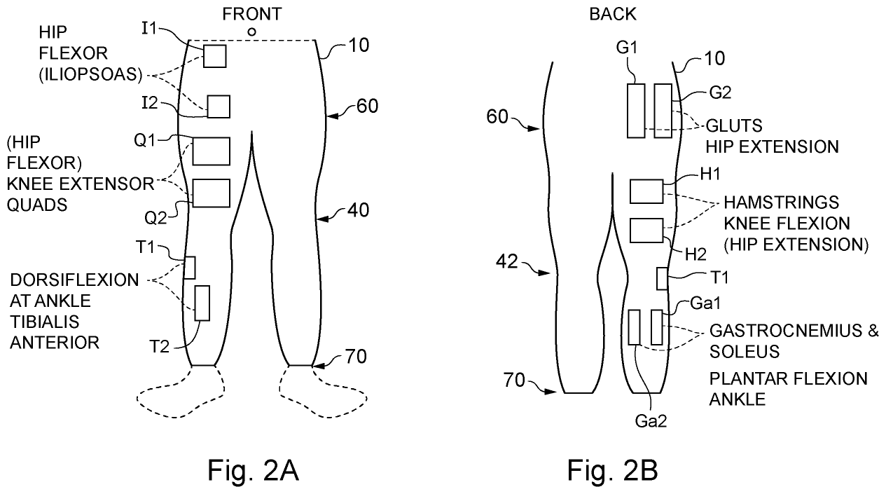

MOINEAU et al in Journal of Rehabilitation and Assistive Technologies Engineering Volume 6:1-15, 2019 describe proof of concept garments for functional electrical stimulation and show knitted electrodes used in leggings (see FIG. 2). There are problems with using such tight garments and in keeping the electrodes comfortable.

MARQUEZ-CHIN and POPOVIC in BioMed Eng OnLine (2020) 19:34 describe functional electrical stimulation therapy for restoration of motor function after spinal cord injury and stroke and highlight problems with knitted textile FES and electrodes in patient comfort.

KAI YANG et al in Sensors and Actuators: A Physical (2014), describes a Screen Printed Fabric Electrode Array for Wearable Functional Electrical Stimulation.

GB2555592B BUNGAY, and WO2020008162A1 and WO2020058660A1 both to BROOK BUNGAY describe conductive electrodes.

BENNETT et al in Proceedings 2019, 32, 17 www.mdpi.com describes a wearable FES compression garment sleeve that uses flexible and extensible screen-printed electrodes.

Gordon PAUL in his 2014 PHD Thesis from the University of Southampton describes screen printed textile-based wearable bio-potential monitoring and refers in section 8.6 to functional electrical stimulation.

The above documents do not describe an easy to wear garment for facilitating functional electrical stimulation, and, in particular, staged functional electrical stimulation, to facilitate motion. Location, orientation, size and arrangement of electrodes all remain challenging problems in providing effective muscle stimulation to elicit or enhance motion, particularly walking, with less fatigue. Known electrodes or devices are frequently uncomfortable, or heavy, or poorly effective, and can often tire muscles quickly. Comfort over time is also a problem. Effectiveness over time is another problem. It is undesirable for a patient to have to use different garments at different stages of their infirmity progression. Provision of a garment with multiple ways of use depending upon a patient's need also remains a problem. Ease of manufacture of such a garment remains a problem.

The present invention seeks to alleviate one or more of the above problems, and other problems of the prior art.

### STATEMENTS OF THE INVENTION

In a first aspect of the invention there is provided an apparatus for facilitating walking in a human body comprising: a garment configured to be worn in a close fit on a leg of a human body, the garment comprising: at least one flexible, articulating portion configured to span a joint of a leg (e.g. one or more of the hip, knee, ankle joints); a flexible, resilient fabric substrate having an inner surface for engaging a leg closely when worn; and, on the inner surface of the substrate, an electrode arrangement comprising: at least two pairs of conductive, flexible, printed electrodes configured with respect to the articulating portion (and so, for example, with respect to the joint of the leg) for electrically stimulating respective, or the same, predetermined muscle group(s) with respect to the joint; further in which the electrode arrangement comprises at least one first pair of electrodes configured to stimulate a first (e.g. the tibialis anterior) muscle group; and, at least one second pair of electrodes configured to stimulate a second (e.g. the quadricep) muscle group or the first muscle group (e.g. a different part of the first muscle group, and/or stimulate the first muscle group in a different way e.g. in a different location and/or orientation and/or direction of the stimulation).

Preferably, the electrode arrangement comprises: on the inner surface of the garment, a pair of electrodes configured to stimulate the tibialis anterior muscle group; on the inner surface of the garment, a pair of electrodes configured to stimulate the quadricep muscle group.

Preferably, the electrode arrangement comprises, on the inner surface of the garment, one or more of:
a pair of electrodes configured to stimulate the hamstring muscle group;
a pair of electrodes configured to stimulate the iliopsoas muscle group;
a pair of electrodes configured to stimulate the gastrocnemius muscle group;
a pair of electrodes configured to stimulate the gluteal muscle group.

Preferably, the electrode arrangement comprises: on the inner surface of the garment, one or more of:
a pair of electrodes configured to stimulate the oblique muscle group associated with the abdominal corset;
a pair of electrodes configured to stimulate the rectus abdominis muscle group associated with the abdominal corset;
a pair of electrodes configured to stimulate the erector spinae muscle group associated with the abdominal corset.

Preferably, one or more of the printed electrodes of the electrode arrangement are one or more of screen printed, stencil printed, inkjet printed, dispenser printed.

Preferably, the apparatus comprises:
optionally, a non-conductive, flexible, printed first layer attached to the fabric substrate in a first pattern;
a conductive, flexible, printed second layer overlaying the fabric substrate and first layer in a second pattern;
a non-conductive, flexible, printed third layer overlaying the fabric substrate, first layer and second layer in a third pattern;

wherein the third pattern defines apertures in the third layer exposing regions of the second layer forming the electrode arrangement of printed electrodes.

Preferably, the apparatus comprises: a conductive, flexible, printed fourth layer overlaying at least in part the exposed regions of the third layer forming the printed electrodes.

Preferably, the second layer comprises a contact trace to each electrode (or to or between each sub electrode where provided), the contact trace encapsulated between the non-conductive first and third layers or between the fabric substrate and the non-conductive third layer.

Preferably, the third pattern defines at least one aperture in the third layer at the end of a contact trace exposing a region of the second layer forming a contact for the electrode.

Preferably, the second layer comprises a first sub-layer of a first conductivity (e.g. forming electrodes) and a second sub-layer of a second conductivity (e.g. forming contract traces known as vias and/or contact pads).

Preferably, at least one of the non-conductive first and third layers comprise a polymeric or rubber or plastic composition. Preferably, the conductive second layer is formed from at least one of gold, silver, silver chloride, copper, carbon printable paste.

Preferably, the electrodes of the electrode arrangement are dry (e.g. in use).

Preferably, the apparatus comprises a control unit for controlling power to the electrode arrangement and configured to supply power to one or more pairs of electrodes. Preferably, the control unit comprises a selection module for selecting one or more pairs of electrodes to be powered.

Preferably, at least one pair of electrodes is provided with a predetermined voltage waveform in a predetermined sequence (e.g. with respect to a gait cycle).

Preferably, at least two pairs of electrodes are each provided with a respective predetermined voltage waveform in a predetermined sequence (e.g. with respect to a gait cycle).

Preferably, the control unit is configured to supply a time varying or alternating current (AC) voltage to at least one pair of electrodes in a predetermined voltage waveform.

The control unit may provide a time varying voltage of any suitable frequency e.g. any range of frequencies for example, 10 Hz to 120 Hz, or 10 Hz to 80 Hz, or 10 Hz to 60 Hz, less than or equal to 60 Hz. In one or more example embodiments the control unit is configured to supply the electrodes with a time varying voltage at ≥60 Hz (greater than or equal to 60 Hz).

Preferably, the fabric substrate is elasticated (e.g. comprises elastic thread).

Preferably, at least one pair of electrodes is arranged so that at least a first electrode of the pair is associated with at least one motor point of a muscle group.

Preferably, at least one pair of electrodes is arranged so that at least one electrode of the pair, optionally a second electrode of the pair, lies along a belly of the muscle group.

Preferably, two pairs of electrodes are provided configured to be associated with one muscle group.

Preferably, the electrode arrangement comprises two pairs of electrodes arranged (generally or substantially) diagonally with respect to one another, a first electrode of one pair diagonally opposite a second electrode of the same pair, a first electrode of one pair (generally or substantially) vertically in line with a second electrode of the other pair.

Preferably, a first pair of (e.g. quadriceps) electrodes is powered on, as (at about the same time or simultaneously) a second pair of (e.g. quadriceps) electrodes is powered off. The two pairs of electrodes may be alternately powered on and off within an overall power waveform for that (e.g. quadriceps) muscle group. There may be an overlap so that, as one pair powers off, the other pair powers on, so that the muscle group is powered for the entire predetermined time period of the waveform, but for just under half the time, each half of the muscle is rested.

Preferably the apparatus comprises one or more of the following:
at least one pair of electrodes inclined with respect to the vertical;
the electrodes of at least one pair of electrodes are inclined with respect to the other to form a V-shape;
at least one electrode is elongate;
at least both electrodes of at least one electrode pair is elongate;
at least one electrode of a pair is generally rectangular;
at least one electrode of a pair is generally square.

In a second aspect of the invention there is provided a method of powering electrodes in an apparatus as described herein comprising:
selecting at least one electrode pair in the garment;
powering the selected at least one electrode pair in the garment in a predetermined sequence (e.g. with respect to a motion cycle).

Preferably, the method comprises:
preparing a flat precursor to a garment;
printing electrodes on the pre-cursor;
assembling the garment.

In a third aspect of the invention there is provided a method of manufacturing an apparatus as described herein comprising: providing any of the features described herein.

In a further aspect of the invention there is provided an apparatus for eliciting or enhancing motion in a human or animal body comprising a garment configured to be worn in a close fit on a body part of a human or animal body; the garment comprising at least a flexible, articulating portion configured to span a joint of a body part [e.g. a finger, shoulder, wrist, arm, leg];
at least a part of the garment (e.g. the flexible, articulating portion) comprising:
a flexible, resilient fabric substrate having an inner surface for engaging a body part closely when worn; and, on the inner surface of the substrate, at least two pairs of [e.g. screen] printed, flexible, conductive electrodes configured with respect to the articulating portion [e.g. with respect to a joint of the body part] each pair configured for electrically stimulating one or more predetermined muscle(s) with respect to the joint, for causing a body part to move.

Several aspects and embodiments of the invention are described in this document, and any one or more features of any one or more embodiments may be used in any one or more aspects of the invention as described above and elsewhere in this document, as would be understood by someone skilled in the art.

### BRIEF DESCRIPTION OF THE INVENTION

The present invention will now be described, by way of example only, with reference to the following figures in which like reference numbers refer to like features.

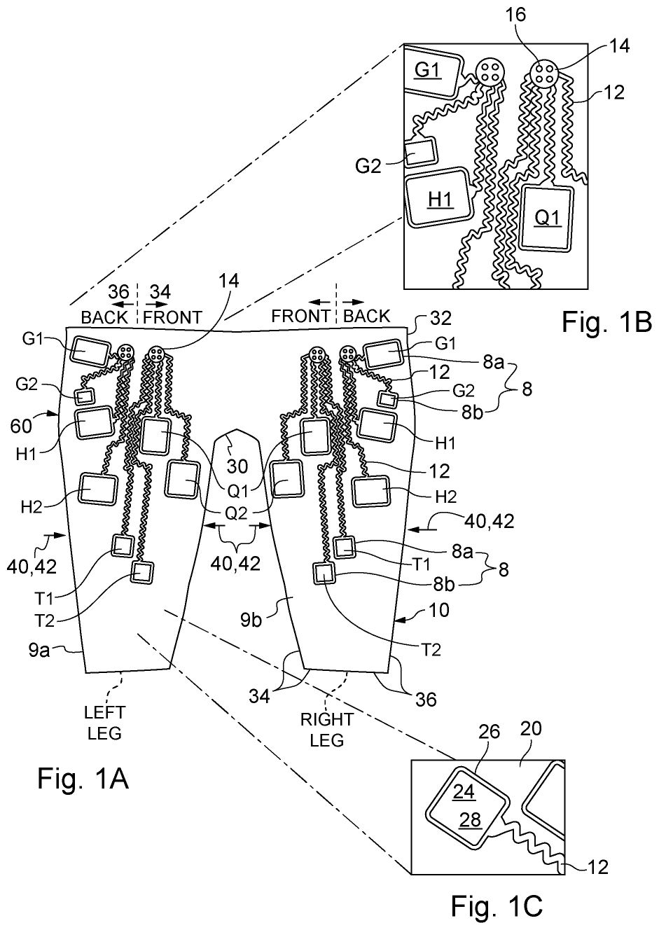

FIG. 1A shows a plan view of a partly constructed garment formed from flexible, resilient fabric, here shown as flat, shaped outline pattern for a garment for a lower part of a human body, here a pair of leggings, suitable for printing and later construction (e.g. sewing to form a complete garment). FIG. 1A shows four pairs of electrodes per side of the garment.

FIGS. 1B and 1C show close-up, plan views of electrodes printed on the fabric substrate of the garment of FIG. 1A.

FIGS. 2A and 2B show, respectively, front and rear (back) views of a garment with six pairs of electrodes per side (electrodes only on one side are shown for simplicity, the right side but, typically, both sides may be provided with the same electrodes).

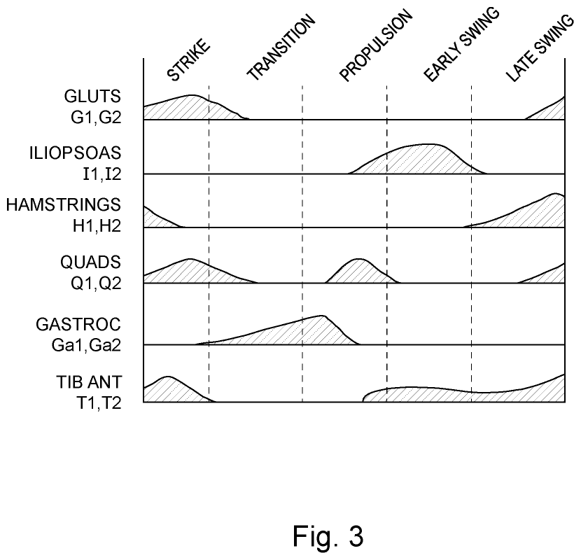

FIG. 3 shows a timing diagram for application of voltage waveforms to particular electrodes, in this case for the six-electrode embodiment of FIGS. 2A and 2B. The horizontal axis is time, which may vary with the speed of the gait cycle i.e. the speed of walking desired, and the vertical axis for each waveform shows the intensity across respective pair(s) of electrodes (G1 and G2, I1 and I2, H1 and H2, Q1 and Q2, Ga1 and Ga2, T1 and T2).

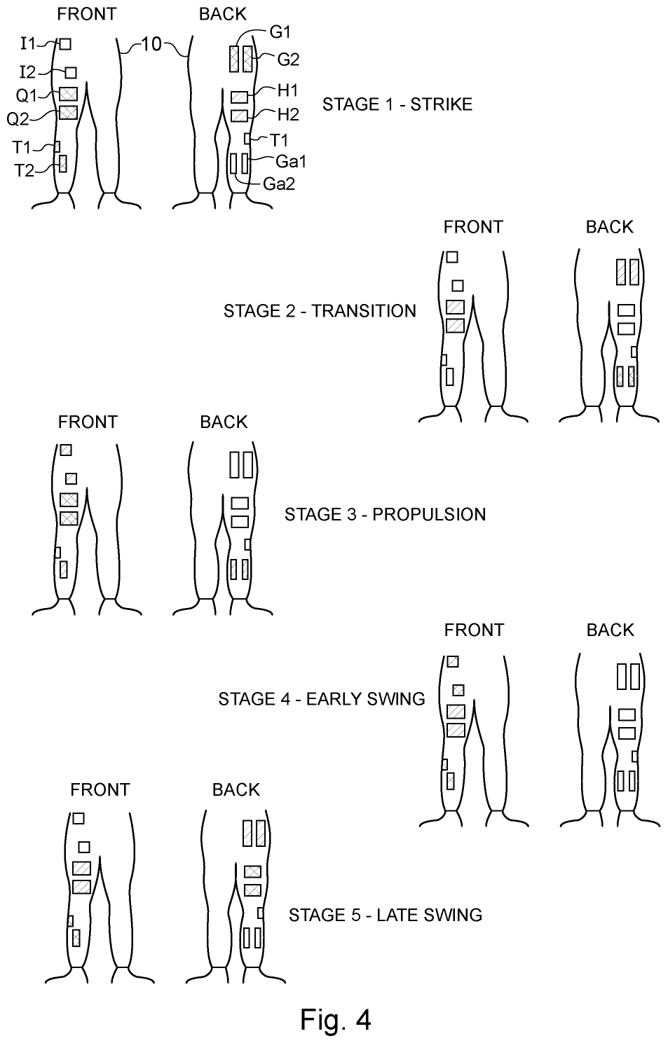

FIG. 4 shows a schematic view of the garment of FIGS. 2A and 2B illustrating the timing of application to the electrode pairs of the voltage waveforms of FIG. 3 in each stage. Cross-hatch indicates a high intensity waveform, single hatch indicates a low or rising or descending intensity waveform and no shading indicates no waveform is applied.

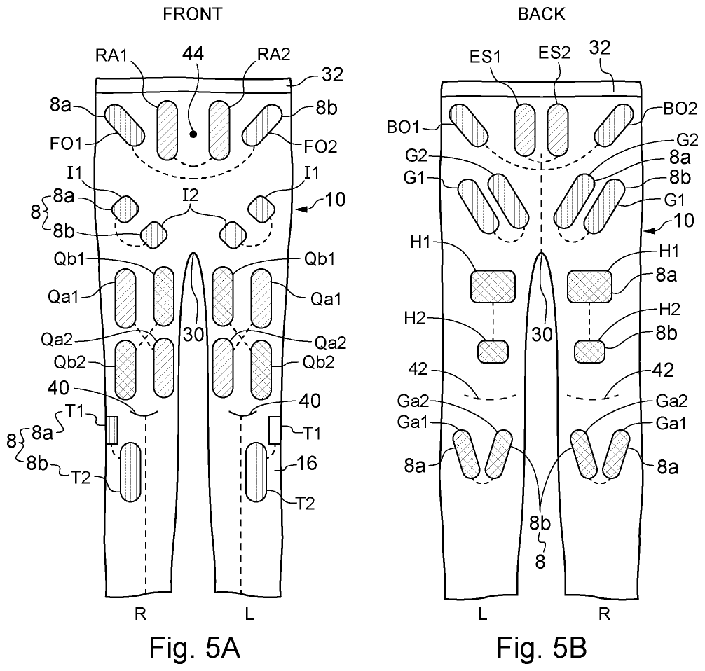

FIGS. 5A and 5B show front and rear (back) views of a garment, here leggings, provided with seven electrode pairs per side and two electrode pairs spanning the abdominal cavity. Not all electrode pairs shown need be provided or, if provided, need be used. One or more electrode pairs may be selected for use.

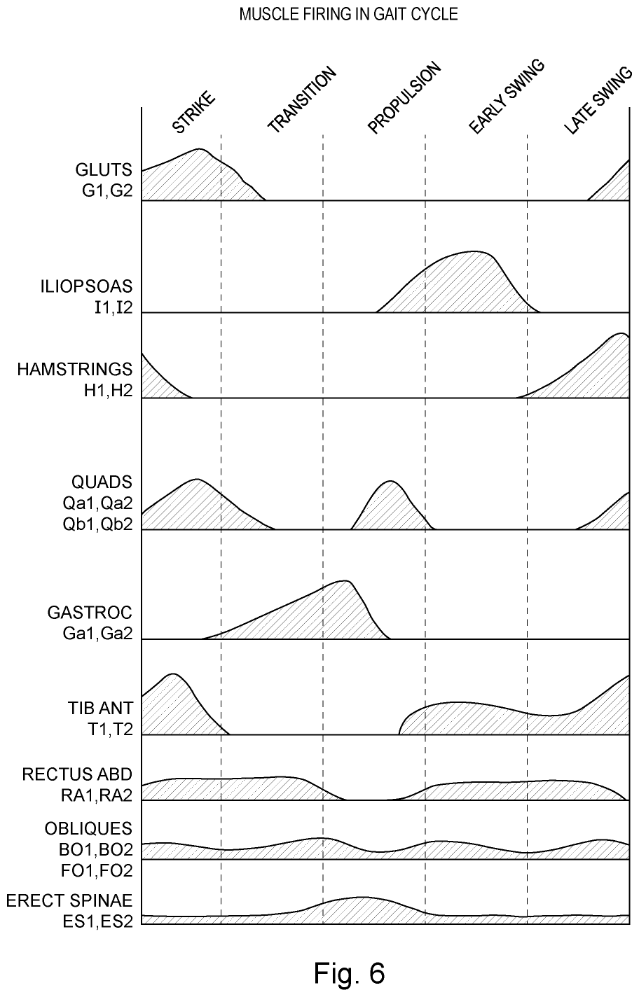

FIG. 6 shows the timing and relative intensities of the muscle firing sequence in the gait cycle for the garment of FIGS. 5A and 5B when all electrodes are in use. Again, timing is along the horizontal axis and intensity is on the vertical axis for each electrode pair (G1 and G2, I1 and I2, H1 and H2, Q1 and Q2, Ga1 and Ga2, T1 and T2, Qa1 and Qa2, Qb1 and Qb2, RA1 and RA2, BO1 and BO2, FO1 and FO2, ES1 and ES2).

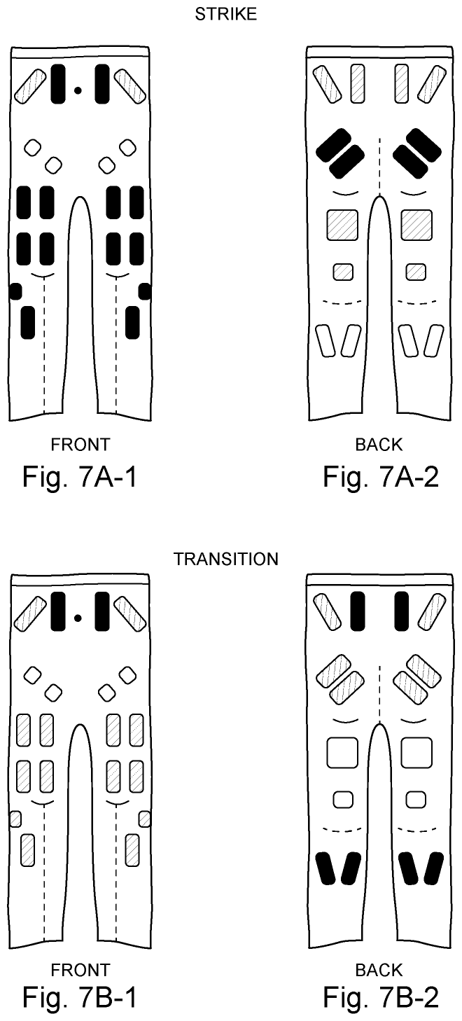

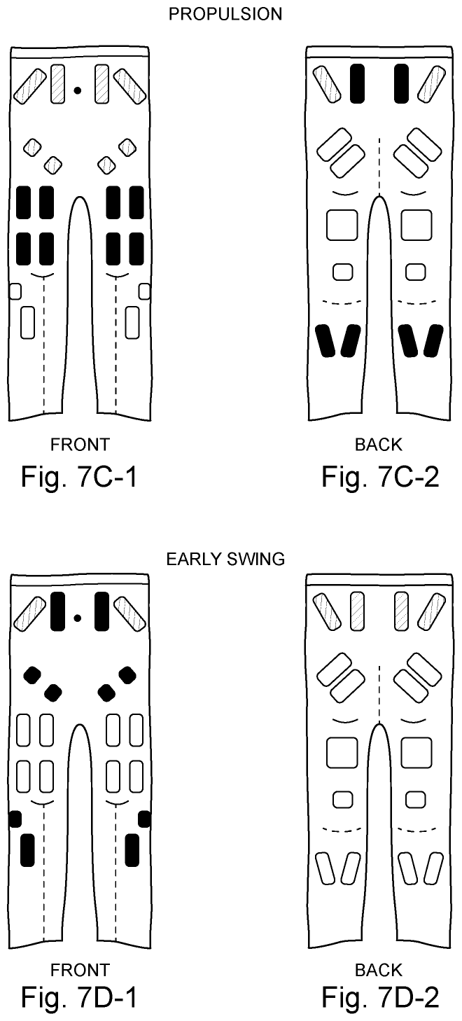

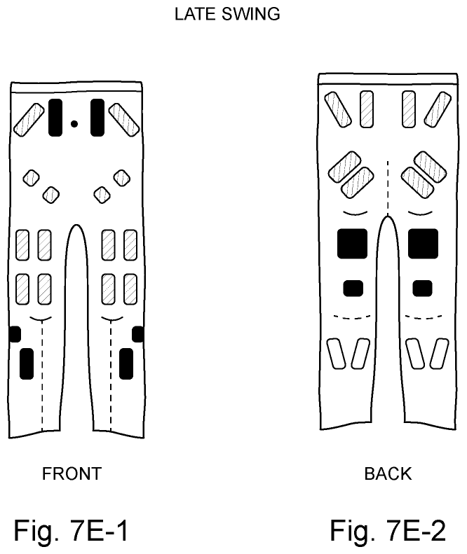

FIG. 7 comprises front (FIG. 7A-1, FIG. 7B-1, FIG. 7C-1, FIG. 7D-1, FIG. 7E-1) and back (rear) (FIG. 7A-2, FIG. 7B-2, FIG. 7C-2, FIG. 7D-2, FIG. 7E-2) views of the garments of FIGS. 5A and 5B at the various staging of the muscle firing sequence shown in FIG. 6. Fully shaded electrodes are operating at high intensity, partially shaded electrodes are at low intensity or rising or falling, and unshaded electrodes are switched off.

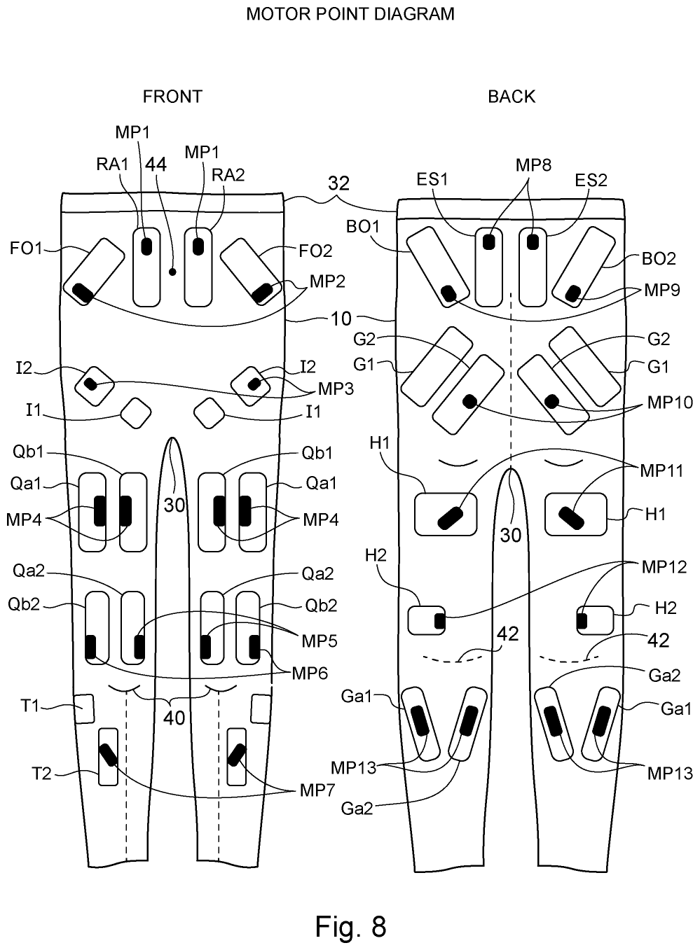

FIG. 8 shows front and rear views of the garment of FIGS. 5A and 5B illustrating the typical position of the motor points for the electrodes shown in FIGS. 5A and 5B. It is noted that, in a further example embodiment of FIG. 8, the front, oblique electrodes in FIG. 8 are inclined upwardly and inwardly towards the centre of the body whereas the back, oblique electrodes are inclined upwardly and outwardly with respect to the centre of the body.

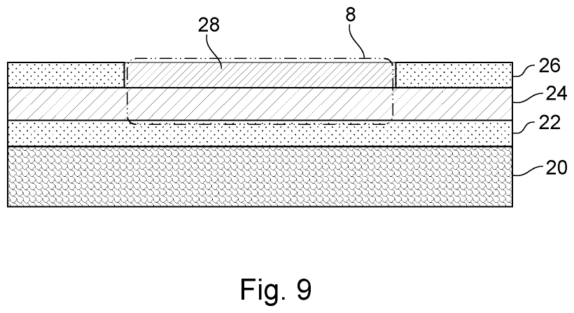

FIG. 9 shows a cross-sectional elevation of a fabric substrate for a garment and associated electrode.

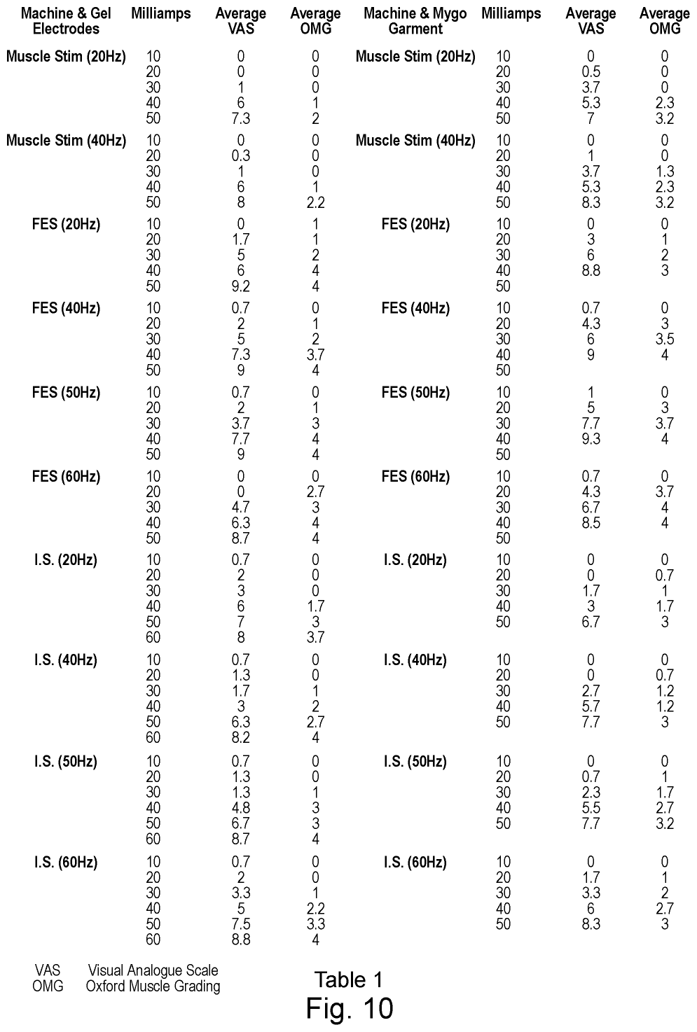

FIG. 10 shows a table of experimental results.

### DETAILED DESCRIPTION OF THE INVENTION

It will be understood by those skilled in the art that any dimensions, relative orientations and configurations such as lower and higher, above, and below, and any directions, such as vertical, horizontal, upper, lower, axial, radial, longitudinal, tangential, flat, resilient elastic, etc., referred to in this application are within expected structural tolerances and limits for the technical field and the apparatus and methods described, and these should be interpreted with this in mind.

In this document, the term resilient is used to describe a fabric substrate or garment which is capable of exerting a force, typically a small force, against the skin of the user when worn. Typically the resilient fabric substrate or garment will be, or will comprise, one or more of: elastic, be elasticated, stretchable, expandable, have a stretchable weave, extensible, or otherwise be provided with a slight resilience to the fabric substrate or garment, so that when worn by a person for which it is sized, a slight stretch able to exert a restoring force occurs. The fabric forming the substrate is typically made from natural and/or man-made fibres. Use of fibres to form the fabric is not essential but is preferred.

In this document a lower body garment such as leggings or tights or trousers (known in the USA as pants) are described, however, the garment may comprise a single tube for a single limb such as a sock or footless legwarmer and such like. Indeed, the garment need not entirely encompass a limb to form a continuous periphery (it may instead be separate panels joined by straps, one or more being resilient) but this is preferred for simplicity of construction. Typically such garments are made from fabric, which comprise yarn, woven or stitched or knotted or otherwise attached together. A 3d printed fabric substrate may also be possible.

In the following document, various layers for providing printed electrodes are described. Strictly speaking it is only the conductive part of the structure which is powered, however, in general usage, the electrode may comprise the associated encapsulating structures which are typically non-conducting that may be used to fix the conducting part to the underlying substrate and define its location, size, orientation and dimensions. Thus, the term electrode may be used to describe the conductive part of the integrated structure or the integrated structure comprising not only the conductive part but also the non-conductive parts. The term electrode is to be read, therefore, in the context of the description or claim in which it is used. On occasion, the integrated structure incorporating a conductive part may be referred to as an encapsulated electrode.

In this document various muscle(s) and muscle group(s) are described. For the sake of simplicity, the full detail of each set of muscle(s) and/or muscle group(s) is not described but it will be understood by those skilled in the art that the overarching term may be used, and this may indicate the entire muscle group or a subset of a muscle group, or set of muscles, or a portion of a muscle or muscle group, as the context indicates. Further, each muscle, set of muscles, or muscle groups are known to act primarily on one joint and may also act on a secondary joint to elicit, or enhance motion. In this document, we typically refer to powering of electrode(s) at an appropriate time for an appropriate duration to elicit the primary action of each muscle, or muscle group, on the primary joint on which these can act. However, powering of these electrodes may be associated with the secondary action of a muscle or muscle group to provide for movement of a secondary joint at an appropriate time during the gait cycle, and for an appropriate duration. This secondary action is not described in detail but will be understood as being a potential variation by those skilled in the art from the information disclosed in this document.

The stimulation provided is typically as an overarching waveform during the gait cycle the waveform comprising pulses with pulse width, amplitude, and frequency which define the force and quality of the contraction. Preferably, the pulses are biphasic (alternating voltage about zero volts) to assist reducing muscle fatigue but monophasic, time varying voltage pulses may be used.

This document refers to the motor point of a muscle group and this represents the location where the motor branch of a nerve enters the muscle belly also known as fasciculi. Whilst the motor point does vary from person to person, and from muscle group to muscle group, these are well-defined anatomically and, if necessary, can be found on a person-by-person basis. Whilst individual motor point identification is possible, it is undesirable in a garment designed for general usage, it will therefore be understood that the anticipated location of the motor point(s) is/are for a range of sizes of person for which that garment is designed. Thus, the expected range of motor point locations for a muscle group for a garment designed for a 2.2 m sized person may be different to the expected motor points of a 1.5 m sized person. Nevertheless, the locations of the motor points for a particular size range of persons can be identified within a predetermined range.

It will also be understood by those skilled in the art that electromechanical stimulation, or EMS, provides for stimulation of muscles for rehabilitation for building strength rather than for motion. The apparatus, including the garment, and methods of the present invention are directed towards functional electrical stimulation, known as FES, to provide stimulated functional motion of a body part, such as a limb or other body part, typically a leg. Typically in FES, firstly, a patient may actively attempt a motor task and, secondly, the FES system produces the intended movement (e.g. eliciting the full motion itself or enhancing a user's own attempt at motion). In this invention, this may be varied from muscle group to muscle group. So one muscle group on one limb (e.g. leg) may provide its one motion, and/or a further muscle group may be fully stimulated into motion, and/or a different muscle group may be partially stimulated.

Thus, in various example embodiments, it is important that the apparatus is capable of determining and engaging with a gait cycle (or other motor cycle of a different body part) of a user, during use, to the extent predetermined by a user, and/or health care professional. Thus, one or more of a power unit, timing circuitry, control circuitry, and selection modules, for powering and controlling the power unit may be provided. These may be configured to provide a predetermined range of intensity of electrical stimulation over time to a variety of selected electrodes pairs, to cause a predetermined, selected amount of muscle stimulation to elicit or enhance motion in the body part to facilitate walking (or such other motion as is desired).

In FIG. 1A, a precursor of a garment 10 is show in plan view on which multiple electrodes 8 arranged in electrode pairs 8a, 8b have been printed as described elsewhere. Each electrode 8 is, in this example embodiment, provided with a conductive trace 12 leading to a contact pad 14 (see close-up in FIG. 1B). In FIGS. 1A and 1B, two pairs of electrodes 8a, 8b are associated with each contact pad 14 so that contact pad 14 has four contacts 16 providing electrical connection to each of the four electrodes 8 associated with that contact pad. These or other combinations of numbers of contacts per contact pad may be used. The garment 10 when fully constructed will form a pair of resilient (i.e. stretchable) leggings with a waistband 32, a crotch 30, an articulated knee portion comprising a knee 40 and knee pit 42, and articulating hip portion 60 (and optionally an articulated ankle portion 70). When sewn up, the two leg portions 9a and 9b form close-fitting resilient tubes into which the legs of a user can be placed. The front of the finished garment is labelled 34 and the back of the finished garment is labelled 36.

Arrows indicate the location of the articulating portions 40, 42, and 60. It will be understood that the garment may comprise an articulating ankle portion 70 in addition to or as an alternative to other articulating portions (see FIG. 2A). Indeed, a single leg garment such as a single tube in the form of a legwarmer (covering ankle portion 70 and knee portion 40, or sock covering ankle portion 70 or both ankle portion 70 and knee portion 40) may be provided.

Referring now to FIGS. 1A, 1B and 1C, electrodes 8 (in pairs comprising first and second electrodes 8a and 8b) are provided in two groups, one on the left side of the garment 10 for the left side of the body and one on the right side of the garment 10 for the right side of the body. One or more pairs of electrodes on each side may be operated, e.g. alternately during walking, or independently e.g. if only one side is needed. A selection module may be provided to facilitate such operation. The dashed vertical lines in FIG. 1A show the fold line along each of the left and right-side portions of garment 10 prior to final sewing. Laying the fabric substrate of garment 10 out flat in this way, as shown in FIG. 1A, enables printing to take place prior to garment construction. This is particularly helpful for manufacture. The fabric of garment 10 is, in this embodiment, elasticated to provide a close-fitting garment when worn. It will be understood that, as an alternative or an addition to elasticating the fabric, the weave or knit or threads used may stretchy, extensible, or otherwise resilient, so that a close fit is formed to the body part, and a slight resultant force from the resilience provides close (typically adjustable) contact between the skin of a user and electrodes 8. The circuitous nature of the contact traces 12 facilitate this stretch and enables a continuous conductive path from the contact 16 to electrodes 8 even when the garment is stretched and/or articulated during motion.

In this example embodiment, on each side of the garment four pairs of electrodes are provided. Typically, at least two pairs of electrodes are provided on each side, preferably including: T1 and T2, respectively the first and second electrodes at the side of the leg portion of the garment for the tibialis anterior muscle group which, when electrically stimulated, provides for dorsiflexion of the ankle; and Q1 and Q2, respectively first and second electrodes at the front upper part of the leg portion of the garment for the quadriceps muscle groups, which, when electrically stimulated, provide for knee extension (as a primary action). The advantage in providing preferably at least two, or preferably more, pairs of electrodes is that, as a user's needs change or develop, a fewer or greater number of pairs of electrodes may be brought into play without requiring a different garment to be supplied. Thus, selection on one or both sides of a subset of electrode pairs from a multiplicity of electrode pairs can be made by a user or a healthcare professional, facilitating optimisation of the functional electrical stimulation for that particular user. Indeed, the garment shown in FIG. 1A provides a simple and elegant solution to stimulate any one or more of a selection of major muscle groups used in walking.

In addition to T1 and T2 and Q1 and Q2, in this example embodiment, garment 10 of FIG. 1A also comprises H1 and H2, first and second electrodes respectively, at the upper rear leg portion of the garment associated with the hamstring muscle group acting as knee flexor(s) (in a primary action). Furthermore, G1 and G2, first and second electrodes respectively, at the rear lower torso part of the garment associated with the gluteal muscle group acting as hip extensor(s) (in a primary action) are also provided, here on portion 36 at the back or rear of garment 10.

Typically, the electrodes are of generally rectangular shape but may be of generally square shape, preferably with rounded corners, or other shapes including circular, oval etc. The shape and orientation and, indeed, number of these may be varied for each muscle group, as will be described later, to enhance muscle action whilst reducing muscle fatigue.

Referring to FIG. 1C and briefly to FIG. 9, the construction of the electrodes 8 in one example embodiment is shown. Fabric substrate 20 is flexible and resilient, in other words stretchable so that, when worn, it provides a close fit to a suitably sized user's body part facilitating close contact between electrode 8 and the skin. Overlaying and, typically directly, attached to fabric substrate 20 is a non-conductive first layer 22 of, for example, flexible polyurethane or other flexible non-conductive material in a first pattern. Typically this is deposited by printing such as screen printing. Alternatively, fabric substrate 20 may already have sufficient insulating properties to prevent electrode 8 from being contacted through fabric substrate 20 such that non-conductive first layer 22 can be omitted. Non-conductive first layer 22 may be attached to fabric substrate 20 by an adhesive (not shown) or other attachment method such as heat pressing. Conductive second layer 24 overlays non-conductive first layer 22 in a second pattern and, depending upon the pattern of each layer, may also overlay fabric substrate 20. Conductive second layer 24 may comprise silver or silver chloride or other conductive material. Typically this is laid down in the form of a printable polymer ink or paste using screen or stencil printing. Thus the non-conductive first layer 22, where provided, is laid down in a first pattern and the conductive second layer 24 is laid down in a second pattern. A non-conductive third layer 26 in a third pattern overlays conductive second layer 24. Depending upon the pattern, non-conductive third layer 26 and fabric substrate 20 may be in direct contact. The layers may comprise sublayers. The layers are typically quite thin to retain the flexibility of the garment when worn.

The printed electrodes are typically not extensible in quite the same way, or to the same extent, as the fabric substrate, but it is advantageous if they are slightly resilient for example comprising at least one resilient component e.g. polyurethane or other plastic or rubber, in, or within the composition of, at least one of the layers forming the electrodes, which adds a certain amount of resilience (ability to stretch before returning to an original shape) to the electrodes. It is also advantageous if the electrodes are flexible (in other words can bend) without being non-resiliently deformable (so that that these return to an original shape when released).

The size and/or shape and/or orientation of the electrodes may be configured to facilitate more effective stimulation of the underlying muscle group as described in this document.

FIG. 2A shows the front and FIG. 2B shows the rear of a garment 10 in use on a person. Garment 10 is sized to be a close fit to the legs of a user. Garment 10 is flexible and resilient and can articulate about the hip along the general line of arrow 60, about the knee along the general line of arrow 40 (which also indicates the knee joint), and along the ankle along the general line of arrow 70. Each side of the garment is provided with six pairs of electrodes. In addition to those shown in FIG. 1, garment 10 in FIG. 2 is provided with electrode I1 and I2, first and second electrodes, respectively, at a lower torso part of the garment for stimulation of the iliopsoas muscle group (a hip flexor) and Ga1, Ga2, first and second electrodes, respectively, at a rear lower leg part of the garment for stimulation of the gastrocnemius muscle group (for plantar flexion of the foot at the ankle). Garment 10 may be provided with a part or full covering of the foot about articulation region 70 at the ankle. Indeed, a sock or single leg covering may be provided as an alternative to a pair of leggings as shown.

FIG. 3 shows the timing (along the x axis) and intensity (along the y axis for each pair of electrodes) of the electrical stimulation applied to each pair of electrodes during a gait cycle for one side of the body. This is shown in further detail in FIG. 4 in which each stage of the gait cycle is illustrated schematically.

Referring now to both FIGS. 3 and 4, in stage one, the heel strike, in other words foot hitting the ground, electrodes G1 and G2 controlling the gluteal muscle group on one side, in this case the right side, of the body are electrically stimulated. Similarly electrodes Q1 and Q2 are electrically stimulated, controlling the quadriceps muscle group for knee extension and rotation. Electrodes H1 and H2 controlling the hamstring muscle group experience a decrease in electrical stimulation and electrodes T1 and T2 controlling the tibialis anterior muscle group experience a strong electrical stimulation to control dorsiflexion at the ankle to raise the foot. In stage 2, the transition, in other words shifting or pivoting of the body over the straight leg and ankle, electrodes G1 and G2 and Q1 and Q2 experience a decrease in electrical stimulation and the electrodes Ga1 and Ga2 experience an increase in electrical stimulation providing plantar flexion of the foot at the ankle. This continues into stage three, propulsion, before decreasing. During this stage, quadriceps electrodes Q1 and Q2 are powered to provide knee extension and some hip extension and the iliopsoas electrodes I1, I2 are stimulated to flex the hip. In the next stage, stage 4 early swing, the iliopsoas electrodes I1 and I2 are stimulated to provide hip flexion and the electrodes T1 and T2, controlling the dorsiflexion at the ankle, continue to be stimulated from the end of the propulsion stage through the early swing stage so that the foot is lifted during this swing phase. In stage 5, late swing, electrodes T1 and T2 continue to be stimulated and, indeed, increase stimulation over the phase to provide increased dorsiflexion at the ankle. Electrodes H1 and H2 controlling the hamstrings are increasingly stimulated to provide for knee flex and assist with hip extension. Towards the end of the late swing, the gluteal electrodes G1 and G2 and the quad electrodes Q1 and Q2 also commence stimulation before the gait cycle is repeated. As will be understood by those skilled in the art, this sequence is repeated in overlapping manner first on one side of the body and then the other. The pairs of electrodes selected to be stimulated, and indeed the degree of stimulation, may be the same on both sides, or varied from side to side, depending on a user's selection.

By providing a multiplicity of pairs of electrodes controlling a number of different muscle groups, the garment may be used by individuals suffering from loss of function in one or more or indeed several muscle groups within the leg. The gait sequence may be initiated by a control unit (not shown) which may incorporate or control a power unit (not shown). The control unit controls the timing and the intensity of the stimulation pulses to the electrodes. Typically it includes a selection module for selecting electrode pairs to activate. The timing may be linked to motion sensors associated with the wearer of the garment optionally located on the garment and/or may be controlled by the wearer of the garment or a healthcare professional, by pre-set or active control circuitry within the control unit. Which electrodes are brought into use may also be selected by the user of the garment and/or the healthcare professional prior to use. Indeed, the garment in the two-legged format shown allows for selection of different electrode pairs on each side of the body to be activated within the gait cycle depending upon the user's particular condition or disability. The intensity, duration and frequency of the stimulation underlying each waveform shown in FIG. 3 may be controlled by the control unit. Preferably, an alternating current (AC) (biphasic) voltage is provided with the overlying intensity of waveform shown in FIG. 4, as this reduces fatigue, although time varying pulses monophasic may be used.

In FIGS. 5A and 5B, seven electrode pairs per side are shown along with two electrode pairs associated with stimulating the abdominal corset. Furthermore, the size, shape and orientation of the electrodes have been improved following experiment. It can be seen, for example, that electrodes of different sizes, shapes, number, and orientations are provided. In FIG. 8, the expected motor point location (or range of locations) for each muscle group is shown with respect to positioning of the electrodes seen in FIGS. 5A and 5B.

The inventors have found that an advantageous way to stimulate a muscle is obtained by placing one electrode at the ‘motor point’, this represents the location where the motor branch of a nerve enters the muscle belly. By stimulating the motor point, better muscle fibre recruitment, with a lower stimulating intensity, is obtained. The other electrode theoretically can be placed anywhere, but in practice the inventors have found that it is best placed along the striations of the muscle fibres, proximally (i.e. on the same muscle) and diagonally on larger muscle groups to minimise muscle fatigue and maximise the area of potential muscle contraction.

Referring to FIGS. 5, 6, 7 and 8 the quadriceps electrodes are provided as two pairs of electrodes, a first pair Qa1, Qa2 and a second pair Qb1, Qb2. In example embodiments of the invention, only these electrodes are provided. In other embodiments, other combinations of electrodes, such as any pf those shown in the figures, is provided.

The dotted lines between each pair of electrodes in FIGS. 5A and 5B are of no physical significance but are a visual aid to indicate the electrode pairings. Thus, for the quadricep electrodes these are arranged in diagonally opposing pairs with Qa1, as shown in FIG. 5A, at the top left and its corresponding second electrode Qa2 is at the bottom right. Similarly, the first electrode of the second pair of quad electrodes Qb1 is at the top right and its cooperating second electrode Qb2 is at the bottom left.

Therefore, instead of one pair of electrodes, two electrode pairs Qa1, Qa2 and Qb1, Qb2 are provided associated with the quadriceps muscle group, each pair arranged diagonally opposing one another. This means that the stimulation can takes place from one electrode, say Qa1, to the other, say Qa2, across the muscle group, and between Qb1 and Qb2, across the muscle group, in a generally diagonally opposite direction as viewed in the Figure. These are usually powered alternately, optionally with power lessoning to one pair say Qa1, Qa2, as it increases to the other pair, say Qb1, Qb2 (e.g. a small overlap, although there may be no overlap).

These electrodes may be used simultaneously but preferably are used in alternating fashion such that within a particular waveform shown in FIG. 6, first quad electrode pair Qa1, Qa2 is triggered then, a short while later, (optionally with an overlap between the rise and fall of the intensity of the electrical stimulus to that first electrode pair) a second electrode pair Qb1, Qb2 is stimulated and so on. In this way, the quadriceps muscle group is stimulated but fatigued less. This alternating between the first electrode pair and a second electrode pair for a single muscle group may occur within the quad muscle firing sequence shown in FIG. 6 (within each peak) or, indeed, one peak such as the strike and transition peak may be applied to the first electrode pair and the propulsion early swing peak may be applied to the second electrode pair.

Qa1, Qa2, Qb1, and Qb2 are typically 5 cm×10 cm in dimension and are generally rectangular and elongate. Typically, these are each orientated with their longitudinal axis along the longitudinal axis of a leg of garment 10. The motor points for the quadricep muscles are shown in FIG. 8 and labelled as MP4 (the proximal motor point rectus femoris (quadriceps)), MP5 (the distal motor point vastus lateralis (quadriceps)), and MP6 (the distal motor point vastus medialis). The sizes and arrangements of the two pairs of quadriceps electrodes Qa1 and Qa2, Qb1 and Qb2 are designed to overlay the expected positions of motor points MP4, MP5 and MP6.

The quadricep muscle group (associated with the Q1 and Q2, or Qa1 and Qa2, Qb1 and Qb2 electrodes) comprising the rectus femoris, the vastus lateralis and the vastus medialis runs from the front of the hip down across the front of the thigh to the top of the tibia. This muscle group straightens and stabilises the knee. This muscle group is on from the end of the late swing to mid-transition and from mid-propulsion to the start of the early swing. By providing diagonal pairs of electrodes such as electrodes Qa1 and Qa2, Qb1 and Qb2 working alternately on and off, muscle fatigue is reduced.

The tibialis anterior muscle group (associated with the T1 and T2 electrodes) runs down the shin vertically on the outside and lifts the foot up by dorsiflexion of the ankle to assist with stepping. Typically, when required, this muscle group is on from the late propulsion through to the strike ending at the early transition stage. Thus, one electrode of electrode pair T1, T2, in this case the lower electrode T2, is somewhat larger and of generally rectangular, elongate orientation along the leg of the garment 10. Typically T1 electrode is 5 cm×5 cm and T2, the lower electrode, is 5 cm×10 m. The lower electrode T2 is sized and oriented such that it lies over the expected position of the motor point MP7 of the tibialis anterior.

The iliopsoas muscle group (associated with the I1 and I2 electrodes) runs diagonally from the front of the pelvis down to the pubic bone. The iliopsoas muscle group flexes the hip in early swing. It is active in late propulsion and finishes at the start of the late swing stage. The electrode pair 11, 12 for the iliopsoas muscle group are generally square of dimensions of or around 5 cm×5 cm and are orientated as diamond shapes with their upper most and lower most apexes lying along a longitudinal axis of a leg of the garment. A line joining the centre of each electrode I1 and I2 would lie inclined at approximately 45° to a central longitudinal axis (typically vertical in use) along the leg. It can be seen in FIG. 8 that the expected motor point position MP3 for the iliacus and psoas muscle groups is expected to lie underneath the position of electrode I2.

FIG. 5A and FIG. 8 show slightly different examples of front oblique electrodes FO1, FO2. In FIG. 5A these are generally rectangular and elongate with a longitudinal axis of the electrode lying at approximately 45° extending outwards and upwards from a longitudinal axis of the garment 10. Alternatively, as seen in FIG. 8, extend inwardly and upwardly, again at approximately 45° to a longitudinal axis of the garment 10. The arrangement in FIG. 8 has shown to be a more effective position for stimulation of the motor point. These are arranged so that they extend over the expected motor point MP2 of the internal oblique muscle group. Electrodes FO1 and FO2 are each typically equidistant from a central location of the garment such as belly button 44.

The rectus abdominis muscle group runs vertically from the bottom of the ribs to the pelvis either side of the tummy button 44. It provides stability around the middle of the torso and helps with spinal flexion and pelvic tilt posteriorly. It is on in the strike and transition and late swing and reduces during propulsion.

Rectus abdominis electrodes RA1 and RA2 are also generally rectangular of approximately 5 cm×10 cm and are oriented with their longitudinal axis, typically, parallel to the central longitudinal axis of the garment 10 positioned equidistant either side of a central point of the garment, for example, either side of the expected position of a belly button 44. Electrodes RA1 and RA2, preferably, lie over respective proximal motor point(s) MP1 for the rectus abdominis muscle group.

The erectus spinae muscle group (associated with the ES1, ES2 electrodes) runs vertically from the mid to the bottom of the spine and provides stability around the middle of the trunk. These muscles anteriorly tilt along the lumbar spine. These muscles are on throughout the gait cycle but increase during the propulsion stage. Electrode pair ES1, ES2 lie on the rear of garment 10, either side of a centre line of the garment extending from crotch 30 to waistband 32 lying over the expected proximal motor point MP8 of the erectus spinae muscle group. A typical size for these electrodes is 5 cm×10 cm. These are typically of generally rectangular shape with a longitudinal axis extending along parallel to a central longitudinal axis of the garment 10.

The internal external obliques (associated with electrodes BO1 and BO2, FO1 and FO2) run from the outside of the ribs on the back to the front of the pelvis diagonally and from the front of the rib cage around to the sacrum at the back of the pelvis diagonally. These muscles stabilise the trunk, in other words the ribs and the pelvis, they are active as background throughout providing the stabilising effect.

Similar to front oblique electrodes FO1 in FIG. 5A, rear oblique electrodes BO1, BO2 are inclined outwardly at approximately 45° to a longitudinal axis extending from the crotch 30 to waistband 32 and equidistant from it. Electrodes BO1 and BO2 are of generally a rectangular construction approximately 5 cm×10 cm and are arranged to overlay expected distal motor point MP9 of the external oblique muscle group.

The gluteals include the gluteus maximus and run from the greater tuberosity of the femur and iliotibial tract to the gluteal surface of ilium, lumbar fascia, sacrum, sacrotuberous ligament.

Referring briefly again to FIG. 5B, this figure shows gluteal electrodes G1 and G2 of generally rectangular shape approximately 5 cm×10 cm in size with a longitudinal axis at approximately 45° to a central longitudinal axis of the garment from crotch 30 to waistband 32, extending upwards and outwards away from this central longitudinal axis spaced apart and parallel to one another. As an alternative, these electrodes may be arranged as shown in FIG. 8, again generally parallel to one another but extending inwardly and upwardly with respect to a central longitudinal axis of the garment. The arrangement of the electrodes in FIG. 8 has shown to be a more effective position to facilitate stimulation of the motor point. Electrode G2 is, in FIG. 8, arranged to extend over the expected position of the motor point MP10 of the gluteus maximus muscle group.

The hamstrings (the biceps femoris, semitendinosus and semimembranosus) run vertically from the back of the hip and pelvis down to the back of the knee to help with extension of the hip and the knee. These are active in late swing through to the mid-strike stage.

In FIG. 8, electrode pair H1, H2 associated with the hamstrings, comprises a larger upper electrode H1 of approximately 12 cm×7 cm in dimension and a lower electrode H2 of approximately 11 cm×6 cm. The upper electrode H1 overlays the expected position of motor point MP11 of the hamstring muscle group comprising the biceps femoris and semitendinosus muscles. Lower electrode H2 overlays the expected motor point position MP12 of the semimembranosus muscles of the hamstrings.

The gastrocnemius (associated with electrodes Ga1 and Ga2) is located at the back of the calf and has two muscle bellies. This muscle group runs from the back of the knee and forms Achilles tendons inserting in to the heel. The gastrocnemius muscle group provides propulsion by plantar flexion at the ankle. These muscles are on at the end of the strike building to early propulsion and reducing towards the end of propulsion.

Electrode pair Ga1, Ga2 are of generally rectangular construction of approximately 5 cm×10 cm in size and have their longitudinal axes arranged in a V-shape, pointing downwardly, either side of a longitudinal axis of a leg. As can be seen from FIG. 8, this arrangement facilitates the location of the electrodes over the expected elongate motor points MP13 of the proximal and distal gastrocnemius muscle groups on the rear of the leg of the user.

FIGS. 6 and 7 (7A-1, 7A-2, 7B-1, 7B-2, 7C-1, 7C-2, 7D-1, 7D-2, 7E-1, 7E-2) are similar to FIGS. 3 and 4 showing the firing sequence of electrode pairs and so associated muscle groups within a gait cycle for the garment shown in FIGS. 5A, 5B and 8. Referring to FIGS. 6 and 7, the muscle firing sequence in the gait cycle for all the electrodes shown in FIG. 7 at various stages is shown. The muscle firing occurs when an associated electrode pair or pairs is stimulated. As will be understood from this disclosure, not all electrodes will be stimulated if a particular individual does not require it, or may not be fully stimulated if the intention is for the individual wearing the garment to have their muscle motion enhanced rather than entirely elicited by powering of suitable electrodes. The control unit may be set to provide the intensity to one or more pairs of electrodes provided in the garment within the gait cycle shown. Typically as a minimum, the electrodes I1, I2 controlling the iliopsoas muscle group may be used to correct foot drop. Although, it will be understood that two pairs of electrodes may be provided for one muscle group as an example embodiment of the invention.

The I1, I2 electrodes are switched on during the propulsion stage, come to full intensity during the early swing, and are switched off during the late swing. The electrodes used for controlling the quadricep muscles e.g. Q1, Q2 in FIG. 1A or 2A or Qa1, Qa2, Qb1, Qb2 in FIG. 5A may be switched on as shown providing an increase in intensity during the strike stage, decreasing in transition, a further electrification during the propulsion phase, switching off in early swing before starting again in late swing. These two muscle groups, the iliopsoas and the quadriceps, are particularly useful in walking and for assisting partially paralysed patients to walk.

The hamstrings electrodes H1, H2, where provided, may be switched on in late swing and switched off during strike and the gluteal electrodes G1 and G2 may also be switched on at the end of late swing, increasing during strike, and switched off during transition.

The gastrocnemius electrodes Ga1 and Ga2 may be switched on during transition, increasing during transition and into the propulsion stage before being switched off before the end of the propulsion stage.

The tibialis anterior electrodes T1 and T2 are switched on towards the end of propulsion and continue at a low level through early swing and late swing before increasing at the end of late swing to a peak during the strike phase and switching off at the end of the strike phase. The stabilising oblique electrodes BO1, BO2, FO1 and FO2 and the spine and torso stabilising electrodes for the rectus abdominis RA1, RA2 and the erectus spinae ES1, ES2 muscle groups are all on throughout with varying intensities, if required.

Table 1 shows the various milliamps (mA) and frequencies (Hz) of the stimulation applied to each electrode within the waveforms shown in FIG. 3 and FIG. 6 and the average visual analogue scale (VAS) for pain and Oxford muscle grading scale (OMG) for motion observed. Experiments (see below) have shown that the dry electrodes in a garment of the invention described herein, perform at least well as existing gel electrodes. The garment is also more wearable than gel electrodes because it is easier to put on and off and to wear (instead of using wet gel electrodes). Further these can be applied by a user themselves, instead of a health care professional by donning a garment of the invention (of a suitable size for that person as described earlier), which in various example embodiments automatically locates the electrodes of the garment in effective locations about the body (preferably in locations associated with one or more respective muscle groups, and various example embodiments in locations associated with the motor points of one or more respective muscle groups. Further a particular range of frequencies has potential to improve user comfort and reduce user fatigue. In particular, whilst lower frequencies may be used, in example embodiments, it is preferred that frequencies of at least 60 Hz are used.

Pilot Experiment Comparing Standard Gel Electrodes with the Mygo Garment (of FIG. 1A)

### Introduction

The aim of this pilot study was to test three different devices capable of delivering stimulation of a single muscle at a range of 20-60 Hz and 0-100 MA in order to investigate the differences between conventional gel electrodes and the Mygo garment of the invention in relation to the quality of muscle contraction and the comfort/tolerance of the stimulation.

### Method

Four test subjects who had no previous neurological disturbances and who all had an understanding of muscle stimulation were used (age range 19-50 2 male 2 female). Each subject applied the electrical stimulation to themselves and documented the results. The Right leg was used in each subject and stimulation was applied to the Tibialis Anterior muscle which is innervated by the Common Peroneal Nerve. Three different on the market machines were used in testing (Appendix 1). Stimulation was applied via the gel electrodes initially using machines 1, 2 then 3 in order. Then re-tested using the garment of the invention.

The strength of muscle contraction was assessed using the Oxford Muscle Grading scale (OMG) (Appendix 2). Pain and tolerance was assessed using the Visual Analogue Scale (VAS) (Appendix 3)

In each test stimulation was applied at the lowest Hz (20 Hz) possible for the machine with milliamps then being increase by 10 MA per test range 0-50 MA). This was then repeated in 10 Hz increments up to the maximum Hz capable from that machine (range 20 Hz-60 Hz). Each test subject completed all tests in one sitting.

### Results

There was comparable data between the use of gel electrodes and the Mygo garment with some evidence that the Mygo garment produced higher pain scores at lower milliamps (mA) than the gel electrodes and the Mygo garment produced a stronger muscle contraction at lower mA than the gel electrodes. The strongest muscle contraction at the lowest mA was found at 60 Hz when using both gel electrodes and the Mygo garment.

### Conclusion

Both existing gel electrodes and the Mygo garment are capable of producing strong muscle contractions within pain tolerance levels when four subjects were tested.

60 Hz and 30-40 MA showed the best results with both gel electrodes and the Mygo garment. Further testing is needed with a larger sample size in order to confirm that the Mygo garment can produce the same muscle contractions within the wearers pain tolerance levels as standard gel electrodes when applying muscle stimulation.

Further testing is needed with multiple muscle groups.

### APPENDIX 1—STIMULATION MACHINES USED

Machine 1 (Muscle Stim in Table 1) Odstock muscle stimulator (microstim 2V2)
Machine 2 (FES in Table 1) Odstock Pace (ODFS Pace)
Machine 3 (IS in Table 1) IntelliSTIM BE-28E Electric Neuromuscular Stimulator

### APPENDIX 2—OXFORD MUSCLE GRADING SCALE (MODIFIED)

(0): No contraction
(1): flicker contraction
(2−): moves through partial Range Of Movement (ROM) gravity eliminated
(2): able to move through full ROM gravity eliminated
(2+): moves through full ROM gravity eliminated with resistance
(3−): partial ROM against gravity
(3): full ROM against gravity
(3+): person hold the position against gravity with slight resistance
(4): full ROM against gravity with moderate resistance
(5): full ROM against gravity with maximum resistance

### APPENDIX 3—VISUAL ANALOGUE SCALE FOR PAIN

| | | | | | |
|---|---|---|---|---|---|
| 0 (No Pain) | 1 | 2 | 3 | 4 | 5 (Moderate Pain) |
| 5 (Moderate Pain) | 6 | 7 | 8 | 9 | 10 (Worst Pain) |

Various embodiments will be apparent to those skilled in the art from the disclosure in this document. For example, multiple pairs of electrodes may be provided per muscle group. Where at least one pair of electrodes is specified, these may comprise multiple sub-electrodes all associated with one side of an electrode pair and stimulated at the same time as all other sub-electrodes of that half the electrode pair. Indeed both sides of an electrode pair may comprise multiple sub electrodes. In particular, features of one aspect, such as the apparatus, may be used in other aspects such as methods of operation of the apparatus and methods of manufacture.
8, 8a, 8b electrodes, first electrode, second electrode of pair
9a, 9b leg portions
10 garment (may be deconstructed e.g. not yet made/sewn up)
12, 12-1, 12-2 conductive trace (e.g. circuitous)
14 contact pad
16 contacts
20 substrate (e.g. flexible, resilient (e.g. elastic) fabric substrate)
22 (optional) non-conductive first layer (e.g. flexible e.g. polyurethane interface layer)
24 conductive second layer (e.g. silver and/or silver chloride printable e.g. polymer ink)
26 non-conductive third layer (e.g. flexible, polyurethane, rubber etc.)
28 (optional) conductive fourth layer (e.g. carbon loaded silicone rubber paste)
30 crotch
32 waistband
34 front
36 back
38 tibial alignment marker (optional)
40 knee (e.g. front articulating portion) or location of knee joint
42 kneepit (e.g. rear articulating portion)
50 waveforms
60 articulating hip portion
70 articulating ankle portion
I1 first electrode, iliopsoas muscle(s)—hip flexor
I2 second electrode, iliopsoas muscle(s)—hip flexor
Q1, Qa1, Qb1 first electrode, quadriceps muscle(s)—knee extensor(s) (and hip flexor(s))
Q2, Qa2, Qb2 second electrode, quadriceps muscle(s)—knee extensor(s) (and hip flexor(s))
T1 first electrode, tibialis anterior muscle(s)—dorsiflexion at ankle
T2 second electrode, tibialis anterior muscle(s)—dorsiflexion at ankle
G1 first electrode, gluteal muscle(s)—hip extensor(s)
G2 second electrode, gluteal muscle(s)—hip extensor(s)
H1 first electrode, ham string muscle(s)—knee flexor(s) (and hip extensor(s))
H2 second electrode, ham string muscle(s)—knee flexor(s) (and hip extensor(s))
Ga1 first electrode, gastrocnemius—plantar flexor of foot at ankle
Ga2 second electrode, gastrocnemius—plantar flexor of foot at ankle
FO1 first electrode, front oblique—abdominal corset muscle(s)
FO2 second electrode, front oblique—abdominal corset muscle(s)
BO1 first electrode, back oblique muscle(s)—abdominal corset muscle(s)
BO2 second electrode, back oblique muscle(s)—abdominal corset muscle(s)
RA1 first electrode, front rectus abdominis muscle(s)—abdominal corset muscle(s)
RA2 second electrode, front rectus abdominis muscle(s)—abdominal corset muscle(s)
ES1 first electrode, erector spinae—abdominal corset muscle(s)
ES2 second electrode, erector spinae—abdominal corset muscle(s)
MP1 proximal motor point rectus abdominis
MP2 motor point internal obliques
MP3 proximal motor point iliacus and psoas
MP4 proximal motor point rectus femoris (quadriceps)
MP5 distal motor point vastus lateralis (quadriceps)
MP6 distal motor point vastus medialis (quadriceps)
MP7 distal and proximal motor point tibialis anterior
MP8 proximal motor point erector spinae
MP9 distal motor point external obliques
MP10 motor point gluteus maximus
MP11 motor point biceps femoris and semitendinosus (hamstrings)
MP12 motor point semimembranosus (hamstrings)
MP13 motor point (proximal and distal) gastrocnemius

1. An apparatus for facilitating walking in a human body comprising:
a garment configured to be worn in a close fit on a leg of a human body, the garment comprising:
at least one flexible, articulating portion configured to span a joint of a leg;
a flexible, resilient fabric substrate having an inner surface for engaging a leg closely when worn; and,
on the inner surface of the substrate, an electrode arrangement comprising:
at least two pairs of conductive, flexible, printed electrodes configured with respect to the articulating portion for electrically stimulating respective, or the same, predetermined muscle group(s) with respect to the joint;
further in which the electrode arrangement comprises at least one first pair of electrodes configured to stimulate a first muscle group; and,
at least one second pair of electrodes configured to stimulate a second muscle group, or the first muscle group.

2. An apparatus according to claim 1 further in which the electrode arrangement comprises:
on the inner surface of the garment, a pair of electrodes configured to stimulate the tibialis anterior muscle group;
on the inner surface of the garment, a pair of electrodes configured to stimulate the quadricep muscle group.

3. An apparatus according to claim 1 in which the electrode arrangement comprises, on the inner surface of the garment, one or more of:
a pair of electrodes configured to stimulate the hamstring muscle group;
a pair of electrodes configured to stimulate the iliopsoas muscle group;
a pair of electrodes configured to stimulate the gastrocnemius muscle group;
a pair of electrodes configured to stimulate the gluteal muscle group.

4. An apparatus according to claim 3 further in which the electrode arrangement comprises:
on the inner surface of the garment, one or more of:
a pair of electrodes configured to stimulate the oblique muscle group associated with the abdominal corset;
a pair of electrodes configured to stimulate the rectus abdominis muscle group associated with the abdominal corset;
a pair of electrodes configured to stimulate the erector spinae muscle group associated with the abdominal corset.

5. An apparatus according to claim 1 in which one or more of the printed electrodes of the electrode arrangement are one or more of screen printed, stencil printed, inkjet printed, dispenser printed.

6. An apparatus according to claim 1 comprising:
optionally, a non-conductive, flexible, printed first layer attached to the fabric substrate in a first pattern;
a conductive, flexible, printed second layer overlaying the fabric substrate and first layer in a second pattern;
a non-conductive, flexible, printed third layer overlaying the fabric substrate, first layer and second layer in a third pattern;
wherein the third pattern defines apertures in the third layer exposing regions of the second layer forming the electrode arrangement of printed electrodes.

7. (canceled)

8. An apparatus according to claim 6 in which the second layer comprises a contact trace to each electrode, the contact trace encapsulated between the non-conductive first and third layers or between the fabric substrate and the non-conductive third layer, and in which the third pattern defines at least one aperture in the third layer at the end of the contact trace exposing a region of the second layer forming a contact for the electrode.

9. (canceled)

10. An apparatus according to claim 6 in which the second layer comprises a first sub-layer of a first conductivity and a second sub-layer of a second conductivity.

11. (canceled)

12. An apparatus according to claim 6 in which the conductive second layer is formed from at least one of gold, silver, silver chloride, copper, carbon printable paste.

13. (canceled)

14. An apparatus according to any preceding claim comprising a control unit for controlling power to the electrode arrangement and configured to supply power to one or more pairs of electrodes, and in which the control unit comprises a selection module for selecting one or more pairs of electrodes to be powered, and in which at least one pair of electrodes is provided, when selected, with a predetermined voltage waveform in a predetermined sequence.

15. (canceled)

16. (canceled)

17. An apparatus according to claim 14 in which at least two pairs of electrodes are each provided with a respective predetermined voltage waveform in a predetermined sequence.

18. An apparatus according to claim 14 in which the control unit is configured to supply a time varying or alternating current (AC) voltage to at least one pair of electrodes in a predetermined voltage waveform.

19. An apparatus according to claim 14 in which the control unit is configured to supply the electrodes with a time varying voltage at ≥60 Hz (greater than or equal to 60 Hz).

20. (canceled)

21. An apparatus according to claim 1 in which at least one pair of electrodes is arranged so that at least a first electrode of the pair is associated with at least one motor point of a muscle group.

22. An apparatus according to claim 1 in which at least one pair of electrodes is arranged so that at least one electrode of the pair, optionally a second electrode of the pair, lies along a belly of the muscle group.

23. (canceled)

24. An apparatus according to claim 1 in which two pairs of electrodes are provided configured to be associated with one muscle group, and in which the electrode arrangement comprises the two pairs of electrodes arranged, generally or substantially, diagonally with respect to one another, a first electrode of one pair diagonally opposite a second electrode of the same pair, a first electrode of one pair, generally or substantially, vertically in line with a second electrode of the other pair.

25. An apparatus according to claim 24 in which a first pair of electrodes is powered on, as a second pair of electrodes is powered off.

26. An apparatus according to claim 1 comprising one or more of the following:
at least one pair of electrodes inclined with respect to the vertical;
the electrodes of at least one pair of electrodes are inclined with respect to the other to form a V-shape;
at least one electrode is elongate;
at least both electrodes of at least one electrode pair is elongate;
at least one electrode of a pair is generally rectangular;
at least one electrode of a pair is generally square.

27. A method of powering electrodes in an apparatus according to claim 1 comprising:
selecting at least one electrode pair in the garment;
powering the selected at least one electrode pair in the garment with a predetermined voltage waveform in a predetermined sequence.

28. A method of manufacturing an apparatus according to claim 1 comprising:
preparing a flat precursor to a garment;
printing electrodes on the pre-cursor;
assembling the garment.

29. (canceled)
